(19) United States
(12) Patent Application Publication
(10) Pub. No.: US 2021/0369948 A1
(43) Pub. Date: Dec. 2, 2021

(54) ANTI-BUBBLE INFUSION DRIP CHAMBER

(71) Applicant: Hankun ZOU, Nanchang (CN)

(72) Inventor: Hankun ZOU, Nanchang (CN)

(21) Appl. No.: 17/207,568

(22) Filed: Mar. 19, 2021

(30) Foreign Application Priority Data
Jan. 19, 2020 (CN) 202010061257.3

(51) Int. Cl. A61M 5/14; A61M 5/36; A61M 5/165

(52) U.S. Cl. CPC A61M 5/1411; A61M 2005/1657; A61M 5/165; A61M 5/36

(57) ABSTRACT

The present invention discloses an anti-bubble infusion drip chamber. The anti-bubble infusion drip chamber comprises a drip chamber main body; the drip chamber main body has an upper portion and a lower portion, namely a drip chamber portion arranged on the upper portion and a filtering portion arranged on the lower portion; the filtering portion is provided with a liquid storage cavity communicated with an inner cavity of the drip chamber portion, and a liquid medicine filter and an air filter which are arranged on two sides of the liquid storage cavity; a liquid medicine flow channel is arranged on the side wall of the filter and is provided with a liquid inlet and a liquid outlet; the liquid inlet is formed above the liquid outlet; a liquid medicine filter membrane is arranged between the liquid inlet of the flow channel and the liquid storage cavity; and an air filter is arranged on the drip chamber main body on at least one side of the filter membrane. A horizontally-placed liquid medicine filter membrane in the traditional drip chamber can cause unsmooth flow of liquid medicine due to surface tension of the liquid medicine and accumulation of impurities. By making an improvement on the drip chamber, the liquid medicine filter membrane is vertically or obliquely arranged, so that on one hand, the impurities can be prevented from being accumulated on the surface of the filter membrane, and on the other hand, the surface tension of the liquid medicine can be destroyed.

### TECHNICAL FIELD

The present invention relates to an infusion drip chamber, in particular to an anti-bubble infusion drip chamber, which belongs to the technical field of medical devices.

### BACKGROUND

At present, an ordinary infusion apparatus commonly used in the medical field is composed of a bottle stopper puncture device protective cover, a bottle stopper puncture device, a short infusion tube, a short exhaust tube, an air filter, an upper dropper, a drip chamber, a flow regulator, a long infusion tube, a liquid medicine filter, a disposable intravenous infusion needle and other components. Such infusion apparatus at least has two problems: 1, bubbles are easily generated in the drip chamber and the tubes, which is easy to cause hazards such as air embolism. 2, Impurities in liquid medicine will enter the human body. Therefore, the drip chamber needs to have self-exhausting and liquid medicine filtering functions.

The Chinese patent CN101791444A discloses a safe non-bubble infusion set, wherein a safety non-bubble assembly is installed at the bottom end of a drip chamber; the safe non-bubble assembly is composed of an ordinary liquid medicine filter membrane, a precision liquid medicine filter membrane and a liquid medicine filter seat; and the ordinary liquid medicine filter membrane and the precision liquid medicine filter membrane are fixed in the liquid medicine filter seat, wherein the ordinary liquid medicine filter membrane is located on the upper layer of the precision liquid medicine filter membrane. Although the above two problems are solved to a certain extent, the horizontally-placed liquid medicine filter membranes will cause unsmooth flow of the liquid medicine due to the influences of the surface tension of the liquid medicine and the accumulation of impurities.

### SUMMARY

An object of the present invention is to provide an anti-bubble infusion drip chamber, so as to solve the above-mentioned problems in the prior art.

The object of the present invention is achieved by the following technical solution.

An anti-bubble infusion drip chamber comprises a drip chamber main body; the drip chamber main body has an upper portion and a lower portion, namely a drip chamber portion arranged on the upper portion and a filtering portion arranged on the lower portion; the filtering portion is provided with a liquid storage cavity communicated with an inner cavity of the drip chamber portion, and a liquid medicine filter and an air filter which are arranged on two sides of the liquid storage cavity; a liquid medicine flow channel is arranged on the side wall of the filter and is provided with a liquid inlet and a liquid outlet; the liquid inlet is formed above the liquid outlet; a liquid medicine filter membrane is arranged between the liquid inlet of the flow channel and the liquid storage cavity; and an air filter is arranged on the drip chamber main body on at least one side of the filter membrane.

Further, the air filter is arranged on the upper side of the liquid storage cavity. Because air is lighter than liquid medicine, it is easier to be exhausted when the liquid medicine flows.

A circle of steps is formed on the end surface inside the liquid medicine filter, and the edge of the liquid medical filter membrane is welded with the steps on the end surface of the filter.

A plurality of supporting protrusions is arranged on the side wall of the liquid medicine filter, and the supporting protrusions are in contact with the filter membrane. Therefore, the filter membrane is presented from sticking to the side wall of the filter under the pressure of the liquid medicine, resulting in deterioration of the filtering effect and reduction in flow rate of the liquid medicine.

The drip chamber portion and the filtering portion are sleeved vertically, and sealed and fixed by means of welding, bonding or hot melting.

The flow channel is a vertical or inclined flow channel.

The air filter is also provided with a sealing cover matched therewith.

Preferably, the top of the drip chamber main body is directly connected with a puncture needle, and an opening in the top of the drip chamber main body is communicated with the puncture needle. The drip chamber main body and a needle seat of the puncture needle are connected by means of clamping, welding or bonding.

The technical solution of the present invention has the following beneficial effects.

1. A horizontally-placed liquid medicine filter membrane in a traditional drip chamber can cause unsmooth flow of liquid medicine due to surface tension of the liquid medicine and accumulation of impurities. By making an improvement on the drip chamber, the liquid medicine filter membrane is vertically or obliquely arranged, so that on one hand, the impurities can be prevented from being accumulated on the surface of the filter membrane, and on the other hand, the surface tension of the liquid medicine can be destroyed.

2. The special design of the liquid medicine flow channel, due to the design of the liquid inlet of the filter, can make the liquid medicine be stored at a predetermined height in the drip chamber.

### BRIEF DESCRIPTION OF THE DRAWINGS

FIG. 1 is a schematic structural diagram of one of central axis surfaces of an embodiment;

FIG. 2 is a schematic structural diagram of one of central axis surfaces of an embodiment;

FIG. 3 is a schematic structural diagram of one of central axis surfaces of an embodiment; and

FIG. 4 is a schematic structural diagram of Embodiment 1 in the present invention.

In drawings, reference symbols represent the following components: 1-drip chamber portion; 2-filtering portion; 3-liquid storage cavity; 4-flow channel; 5-liquid inlet; 6-liquid outlet; 7-liquid medicine filter membrane; 8-supporting protrusion; 9-air filter.

### DETAILED DESCRIPTION

The technical features of the present invention are further described below in conjunction with the accompanying drawings and specific embodiments.

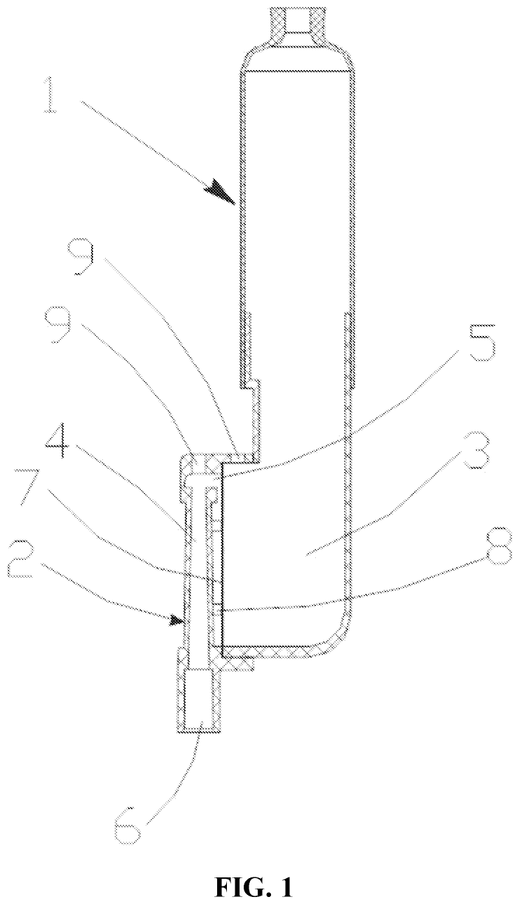

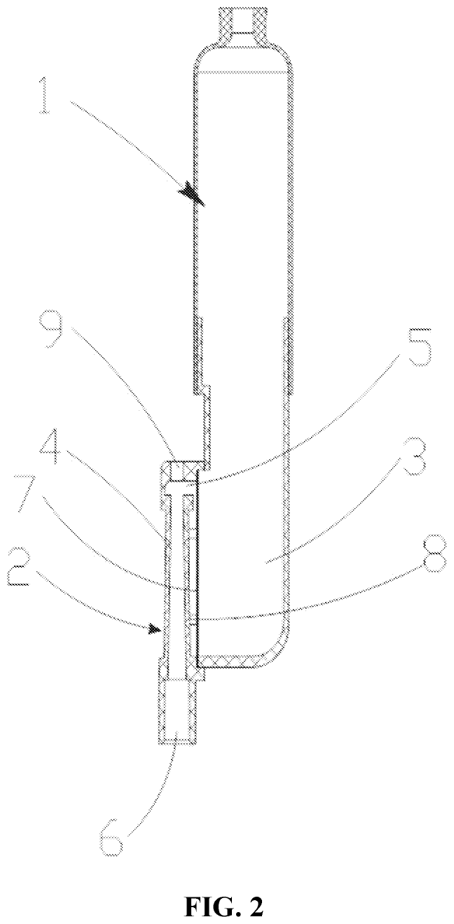

An anti-bubble infusion drip chamber, as shown in FIG. 1 and FIG. 2, comprises a drip chamber main body; the drip chamber main body has an upper portion and a lower portion, namely a drip chamber portion 1 arranged on the upper portion and a filtering portion 2 arranged on the lower portion; the filtering portion 2 is provided with a liquid storage cavity 3 communicated with an inner cavity of the drip chamber portion 1, and a liquid medicine filter and an air filter 9 which are arranged on two sides of the liquid storage cavity 3; a liquid medicine flow channel 4 is arranged on the side wall of the liquid medicine filter and is provided with a liquid inlet 5 and a liquid outlet 6; the liquid inlet 5 is formed above the liquid outlet 6; a liquid medicine filter membrane 7 is arranged between the liquid inlet 5 of the flow channel 4 and the liquid storage cavity 3; and the air filter 9 is arranged on the drip chamber main body on at least one side of the filter membrane. As shown in FIG. 1, the air filter is respectively arranged on two sides of the filter membrane 7. The air filters arranged on two sides can effectively exhaust air in the liquid storage cavity 3 and the flow channel 4. As shown in FIG. 2, an air filter is also arranged outside the filter membrane 7.

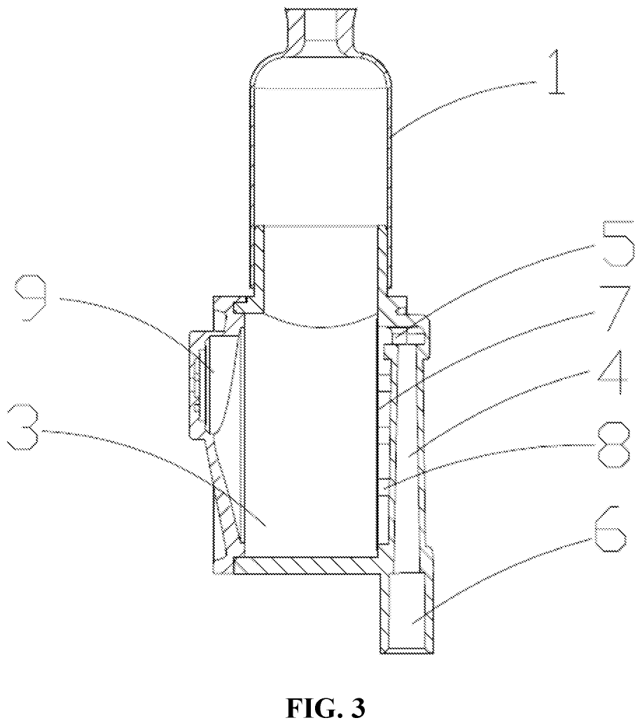

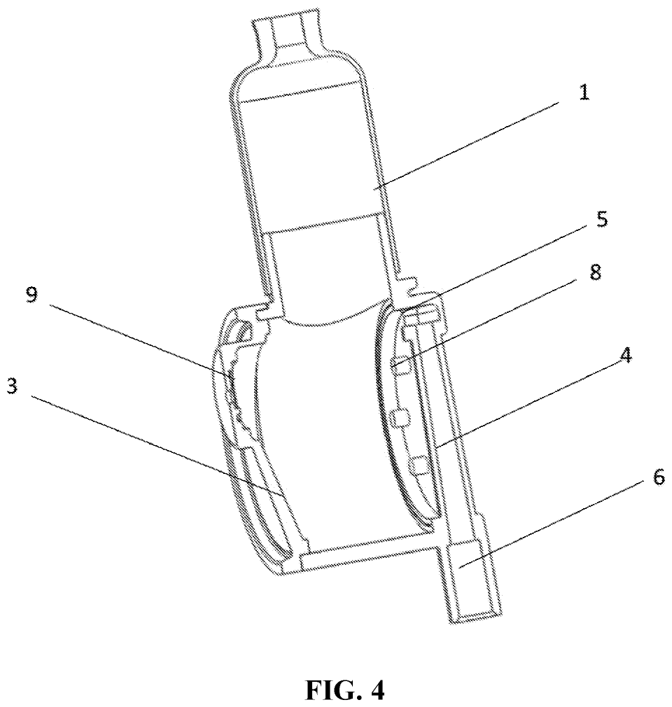

As shown in FIG. 3 and FIG. 4, an air filter is mounted on the liquid storage cavity 3 on the opposite side of the flow channel 4.

A circle of steps is formed on the end surface inside the liquid medicine filter, and the edge of the liquid medical filter membrane 7 is welded with the steps on the end surface of the filter.

A plurality of supporting protrusions 8 is arranged on the side wall of the liquid medicine filter, and the supporting protrusions 8 are in contact with the filter membrane 7. Therefore, the liquid medicine filter membrane 7 is presented from sticking to the side wall of the liquid medicine filter under the pressure of the liquid medicine, resulting in deterioration of the filtering effect and reduction in flow rate of the liquid medicine.

The drip chamber portion 1 and the filtering portion 2 are sleeved vertically, and sealed and fixed by means of welding, bonding or hot melting.

The flow channel 4 is a vertical or inclined flow channel 4.

The air filter 9 is also provided with a sealing cover matched therewith.

During use, the drip chamber portion 1 is communicated with the liquid medicine. After the liquid medicine enters the drip chamber main body, since the liquid inlet 5 is relatively high, the liquid medicine accumulates in the liquid storage cavity 3 and the liquid level rises until the liquid level of the liquid medicine exceeds the height of the liquid inlet 5. At this time, the liquid medicine enters the flow channel 4 from the liquid inlet 5, and finally flows out of the drip chamber main body at the liquid outlet 6.

During use, the liquid medicine passing through the liquid medicine filter membrane 7 before reaching the liquid inlet 5 from the liquid storage chamber 3. Different from a horizontally-placed liquid medicine filter membrane in the prior art, the liquid medicine filter membrane 7 of the present invention has a vertical or inclined design. Therefore, the problem that the liquid medicine cannot flow through the liquid medicine filter membrane smoothly due to surface tension of the liquid medicine and accumulation of impurities is avoided in the filtering process.

In an embodiment with a puncture needle, the top of the drip chamber main body is directly connected with the puncture needle, and an opening in the top of the drip chamber main body is communicated with the puncture needle. In the traditional method, the puncture needle is connected to the drip chamber main body through a tube. Compared with the traditional method, the technical solution of the present invention can avoid entanglement of tubes and reduce connection points to ensure the air tightness.

What is claimed is:

1. An anti-bubble infusion drip chamber, comprising a drip chamber main body; the drip chamber main body has an upper portion and a lower portion, namely a drip chamber portion arranged on the upper portion and a filtering portion arranged on the lower portion; the filtering portion is provided with a liquid storage cavity communicated with an inner cavity of the drip chamber portion, and a liquid medicine filter and an air filter which are arranged on two sides of the liquid storage cavity; a liquid medicine flow channel is arranged on the side wall of the filter and is provided with a liquid inlet and a liquid outlet; the liquid inlet is formed above the liquid outlet; a liquid medicine filter membrane is arranged between the liquid inlet of the flow channel and the liquid storage cavity; and an air filter is arranged on the drip chamber main body on at least one side of the filter membrane.

2. The anti-bubble infusion drip chamber according to claim 1, wherein a circle of steps is formed on the end surface inside the liquid medicine filter, and the edge of the liquid medical filter membrane is welded with the steps on the end surface of the filter.

3. The anti-bubble infusion drip chamber according to claim 1, wherein a plurality of supporting protrusions is arranged on the side wall of the liquid medicine filter, and the supporting protrusions are in contact with the filter membrane.

4. The anti-bubble infusion drip chamber according to claim 1, wherein the drip chamber portion and the filtering portion are sleeved vertically, and sealed and fixed by means of welding, bonding or hot melting.

5. The anti-bubble infusion drip chamber according to claim 1, wherein the flow channel is a vertical or inclined flow channel.

6. The anti-bubble infusion drip chamber according to claim 1, wherein the air filter is also provided with a sealing cover matched therewith.

7. The anti-bubble infusion drip chamber according to claim 1, wherein the air filter is arranged on the upper side of the liquid storage cavity.

8. The anti-bubble infusion drip chamber according to claim 1, wherein the top of the drip chamber main body is directly connected with a puncture needle, and an opening in the top of the drip chamber main body is communicated with the puncture needle.

9. The anti-bubble infusion drip chamber according to claim 8, wherein the drip chamber main body and a needle seat of the puncture needle are connected by means of clamping, welding or bonding.
